(19) United States
(12) Patent Application Publication
(10) Pub. No.: US 2006/0234931 A1
(43) Pub. Date: Oct. 19, 2006

(54) Treatment of diseases with kinase inhibitors

(76) Inventors: William H. Biggs III, San Clemente, CA (US); Todd Carter, San Diego, CA (US); Miles A. Fabian, La Jolla, CA (US); David J. Lockhart, Del Mar, CA (US); Patrick Parvis Zarrinkar, San Diego, CA (US); Daniel Kelly Treiber, San Diego, CA (US); Phillip Edeen, Poway, CA (US)

Correspondence Address: WILSON SONSINI GOODRICH & ROSATI, 650 PAGE MILL ROAD, PALO ALTO, CA, 94304-1050, US

(21) Appl. No.: 10/894,877

(22) Filed: Jul. 19, 2004

Related U.S. Application Data
(60) Provisional application No. 60/488,513, filed on Jul. 17, 2003.

(51) Int. Cl. A61K 38/54

(52) U.S. Cl. 514/12

(57) ABSTRACT

The invention is directed to the identification and use of additional targets of BIRB 796, imatinib mesylate, and BAY 43-9006. The new targets of BIRB 796, imatinib mesylate, and BAY 43-9006 can be used to screen for suitable therapeutic compounds. Also, novel therapeutic and prophylactic uses for BIRB 796, imatinib mesylate, and BAY 43-9006 are disclosed herein.

### CROSS-REFERENCE TO RELATED APPLICATIONS

This application claims the benefit of U.S. Provisional Application No. 60/488,513 filed Jul. 17, 2003, all of which is incorporated herein by reference.

### BACKGROUND

BIRB 796, from Boehringer Ingelheim Pharmaceuticals, Ridgefield, Conn. is an anti-inflammatory agent that binds p38 mitogen-activated protein (MAP) kinase (p38/MAPK14), a serine-threonine protein kinase. BIRB 796 is used as an inhibitor of p38/MAPK14, which regulates production of several proinflammatory cytokines, including tumor necrosis factor-(TNF-α) and interleukin-1β. Excess production of these cytokines is associated with various inflammatory conditions.

The well-known drugs Enbrel (Immunex and Wyeth) and Remicade (Centocor) reduce circulating levels of soluble TNF and have been used against rheumatoid arthritis and Crohn's disease. The structure and mechanism of BIRB 796 are provided by Pargellis et al. (Nat. Struct. Biol., 9(4):268 (2002)). After BIRB 796 binds an allosteric binding pocket of the kinase, ATP no longer binds, thus blocking kinase activity. BIRB 796 has also been described as inhibiting kinase activation.

BIRB 796 has entered and completed Phase I and II clinical trials and is now in Phase III clinical trials for psoriasis. BIRB 796 has also been identified as binding c-Jun kinase 2 (aka mitogen-activated protein kinase 9 or JNK2/MAPK9), Fyn tyrosine kinase, and lymphocyte specific protein-tyrosine kinase (LCK kinase).

Gleevec (imatinib mesylate from Novartis, also known as STI-571) inhibits protein tyrosine kinases. Gleevec is used to treat patients with Philadelphia chromosome-positive chronic myelogenous leukemia (CML) and gastrointestinal stromal tumors (GISTs). In CML, the Philadelphia chromosome creates an abnormal tyrosine kinase (BCR-ABL wherein Abl refers to Abelson tyrosine kinase) which is inhibited by Gleevec binding such that the transfer of phosphate to substrates by BCR-ABL is reduced. This halts the proliferation of BCR-ABL positive white blood cells and induces apoptosis. Gleevec is also used to treat patients with c-Kit tyrosine kinase (CD 117) positive gastrointestinal stromal tumors (GIST) and is known as an inhibitor of platelet-derived growth factor (PDGF) α- and β-receptors (Bohmer et al. J. Biol. Chem. 278(7):5148-55, 2002).

Compound BAY 43-9006 from Bayer Pharmaceuticals Corp. and Onyx Pharmaceuticals, Inc. is an inhibitor of Raf kinases which function in the Ras signaling pathway in many cancers. A particular Raf kinase, BRAF, is mutated in many cancers. BAY 43-9006 has been observed to be effective against liver cancer (hepatocellular carcinoma) and kidney cancer. It has also been used to treat patients with melanoma as well as ovarian, pancreatic, colorectal, nasopharyngeal, esophageal, gastric, liposarcoma, and mesothelioma tumors. BAY 43-9006 has also been used in combination therapy with gemcitabine, carboplatin, irinotecan, vinorelbine, and paclitaxel.

Mutations in the Abl tyrosine kinase have been identified as associated with Gleevec (imatinib mesylate) resistant forms of leukemia. See WO 02/102976; Gorre et al. (2001) Science 293:876-880; Science 293:2163a, Sep. 21, 2001; and Roumiantsev et al. (2002) Proc. Natl. Acad. Sci., USA 99(16):10700-10705.

The lymphocyte specific protein-tyrosine kinase (LCK kinase or p56^{lck} kinase) has been used as a target for the treatment of inflammation and the induction of immunosuppression.

Platelet-derived growth factor (PDGF) receptor (PDGFR) and the vascular endothelial growth factor (VEGF) receptor-2 (VEGFR2 also known as Kinase insert Domain containing Receptor or KDR), both tyrosine kinases, have been used in relation to studies on inhibiting angiogenesis and neovasculature (Bergers et al. J. Clin. Invest. 11(9):1287-95, 2003; and Patel et al. J. Pharmcol. Exp. Therp. E-publication on May 23, 2003 as DOI: 10.1124/jpet. 103.052167); treatment of cancer, such as osteosarcoma (McGary et al. Clin. Cancer Res. 8(11):3584-91, 2002), small cell lung cancer (Abrams et al. Mol. Cancer Ther. 2(5):471-8, 2003), angiomyolipoma and neoplasms associated with tuberous sclerosis (Arbiser et al. Am. J. Pathol. 161(3):781-6, 2002), and myeloproliferative disease (Apperley et al. N. Engl. J. Med. 347(7):481-7, 2002); as well as inhibition of smooth cell proliferation, intimal hyperplasia, and restenosis, including that associated with vascular grafts (Gazit et al. Bioorg. Med. Chem. 11(9):2007-18, 2003; and Karck et al. Transpl. 74(9):1335-41, 2002).

The citation of references herein is not intended as an admission that any of the foregoing is pertinent prior art. All statements as to the date or representation as to the contents of documents herein is based on the information available to the applicant and does not constitute any admission as to the correctness of the dates or contents of these documents.

### SUMMARY OF THE INVENTION

The present invention is based upon the discovery of additional cellular targets for particular protein kinase inhibitors. Additional targets for BIRB 796, Gleevec (imatinib mesylate), and BAY 43-9006 have been identified.

One of the additional cellular targets of BIRB 796 is the Thr334Ile (also known as Thr315Ile) mutant of Abl tyrosine kinase, which is a frequently found mutation associated with Gleevec resistance. In one aspect of the invention, BIRB 796 is used for the modulation of Thr334Ile mutant of Abl tyrosine kinase. In some embodiments, BIRB 796 is used for the treatment and/or prevention of diseases mediated by Thr334Ile mutant of Abl tyrosine kinase, such as Gleevec resistant leukemia.

One of the additional cellular targets of Gleevec (imatinib mesylate) is lymphocyte specific protein-tyrosine kinase (LCK kinase or p56^{lck} kinase). In another aspect of the invention, Gleevec is used for the modulation of LCK kinase. In some embodiments, Gleevec is used in the treatment or prevention of diseases mediated by LCK kinase, such as inflammation, autoimmune disorders, and for the induction of immunosuppression.

Some of the additional cellular targets of BAY 43-9006 are p38 mitogen-activated protein (MAP) kinase (p38/MAPK14), Gleevec resistant and sensitive Abl kinases, the platelet-derived growth factor (PDGF) receptor (PDGFR), and the vascular endothelial growth factor (VEGF) receptor-2 (VEGFR2). In one aspect of the invention, BAY 43-9006 is used in the modulation of p38/MAPK14, Gleevec resistant and sensitive Abl kinases, PDGFR, and VEGFR2, or a combination thereof BAY 43-9006 may be used for the inhibition or prevention of diseases mediated by p38/MAPK14, Gleevec resistant and sensitive Abl kinases, PDGFR, and VEGFR2. Such disease include, but are not limited to, proinflammatory cytokine production, such as TNF-α and interleukin-1β and the treatment or prevention of inflammation and various inflammatory conditions, such as but not limited to, psoriasis, rheumatoid arthritis, and Crohn's disease. BAY 43-9006 may also be used to inhibit c-Abl kinase and thus treat or prevent chronic meylogenous leukemia (CML) as well as Gleevec resistant CML.

BAY 43-9006 may also be used in the inhibition and/or prevention of angiogenesis and neovasculature, particularly in the context of treating or preventing various cancers, such as, but not limited to, solid tumors, metastasized tumors, osteosarcoma, small cell lung cancer, angiomyolipoma, neoplasms associated with tuberous sclerosis, and myeloproliferative disease. Thus, BAY 43-9006 may be used to inhibit the proliferation of tumor vasculature and reduce interstitial pressure in tumors. These uses of BAY 43-9006 may occur with or without the inhibition of Raf kinase.

BAY 43-9006 may also be used to inhibit or prevent smooth cell proliferation, intimal hyperplasia, and restenosis, including that associated with vascular grafts. Furthermore, BAY 43-9006 may be used in the inhibition of VEGFR2 mediated vascular permeability to treat or prevent the loss of visual acuity in diabetic retinopathy (DR) as well as neovasculature related macular degeneration, including choroidal neovasculature (CNV) mediated age-related macular degeneration (AMD), ocular edema, and ocular or retinal neovasculature.

The invention also provides assays for the identification of additional compounds that bind the identified targets, preferably selectively over binding to other cellular factors. Additional compounds that bind these targets may be used to produce the same effect(s) as BIRB 796, imatinib mesylate, and BAY 43-9006 on the targets, upon administration to a subject, as well as to treat and/or prevent diseases and unwanted conditions. Non-limiting examples of BIRB 796, imatinib mesylate, and BAY 43-9006 action mediated by the targets include, but are not limited to, those known from the use of these agents as described herein.

In one aspect of the invention, methods for the identification of additional compounds that bind one or more of the additional targets of BIRB 796, imatinib mesylate, and/or BAY 43-9006 are provided. The methods are screening assays that rely upon the identity of a target and the ability to detect the result(s) of a binding event to a target. The detection of a binding event may be made directly or indirectly, and identifies a compound as capable of binding a target. The compound may be any chemical agent, including small molecules. Preferably, the identified compounds bind a target with a K_{d} less than a range from about 1 μM to 10 nM and/or are selective for said target. Compounds that bind potentially have activities like those mediated by BIRB 796, imatinib mesylate, and/or BAY 43-9006 action in the prevention or treatment of diseases and unwanted conditions. Preferably, the assays are conducted under quantitative conditions such that the affinity, or relative affinity, of binding of a compound to a target may be determined.

In one embodiment of the invention, the assays are based upon the expression of a target on the surface of phage particles that is contacted with a test compound followed by detection of binding between the target and the compound. In a preferred form, the contacting may be made in the presence of another compound that binds the target, such as BIRB 796, imatinib mesylate, and/or BAY 43-9006, and thus may be based upon the ability of a test compound to compete with BIRB 796, imatinib mesylate, and/or BAY 43-9006 for binding to the target. In a preferred embodiment, additional compounds that bind a target are identified by the use of screening methods as disclosed in copending U.S. patent application Ser. No. 10/115,442, filed 2 Apr. 2002, and Ser. No. 10/406,797 filed on 2 Apr. 2003 (or PCT International Application PCT/US03/10247 filed 2 Apr. 2003), both of which are incorporated by reference as if fully set forth.

A test compound of the invention maybe a member of a class of compounds such that all members of the class may be tested for binding to the targets. The assaying of a class of compounds permits the identification of the selective binding of one or some members of the class, as opposed to other members of the class, as binding the targets. This may be used to identify the binding members of the class as more selective for the targets, or alternatively, the non-binding members of the class as preferentially non-selective for the targets. As a non-limiting example, kinase inhibitor compounds in addition to BIRB 796, imatinib mesylate, and BAY 43-9006 may be used in the practice of the invention to identify whether they bind the targets to determine whether they are capable of mediating the same action as BIRB 796, imatinib mesylate, and/or BAY 43-9006 binding or whether they do not bind.

In another embodiment, the invention provides methods of identifying or screening for additional compounds that decrease (inhibit) the function and/or activity of one or more target polypeptides or fragments, portions, or analogs thereof. The methods may be performed in vitro or in vivo. One method for identifying a compound as binding and inhibiting the activity of a target comprises providing an indicator composition comprising a target polypeptide or fragment, portion, or analog thereof, contacting the indicator composition with a test compound (a potential LCK kinase, p38/MAPK14, imatinib mesylate resistant and sensitive Abl kinases, PDGFR, and/or VEGFR2 inhibitor), and determining the effect of the test compound on target activity in the indicator composition to identify a compound that inhibits the activity or function of the target. The methods are preferably used to identify inhibitors for use in the treatment or prevention of diseases and unwanted conditions as disclosed herein.

In another aspect of the invention, the compounds identified by the methods of the invention to bind a target are used to treat or prevent conditions as mediated by BIRB 796, imatinib mesylate, and/or BAY 43-9006 action in vivo. In some embodiments, a compound affects the function and/or activity of a target such that the compound may be administered to a subject, preferably human, in need of a change in the function and/or activity of the target. The invention thus provides for the treatment of a disease or undesirable condition mediated by unwanted or excess target activity, including the binding of a target to its binding partner(s) or its association with other protein(s). The compounds of the invention are expected to include those useful for the modulation of cellular signaling cascades mediated by LCK kinase, p38/MAPK14, imatinib mesylate resistant and sensitive Abl kinases, PDGFR, and/or VEGFR2 as well as those for the treatment or prevention of cancer and other diseases.

The administration of a compound of the invention may be by any appropriate means known in the field, including systemic and localized administration. Prior to administration, the compounds may be formulated as compositions suitable for pharmaceutical or clinical use. Such compositions may comprise appropriate carriers or excipients, such as those for topical, inhalation, or systemic administration.

In yet another aspect, the invention provides methods for determining the level of inhibition by a target binding compound in the treatment of a disease or unwanted condition. Such methods include the administration of a target binding compound to a subject followed by determination of the level of inhibition mediated by said compound in comparison to a subject who has not been administered said compound or to a subject that has been administered a different amount or concentration of said compound. The level of inhibition may be determined by the efficacy of the compound in the treatment of the disease or unwanted condition. Alternatively, the level of inhibition may be determined by the inhibition of a phenotype mediated by the target of said compound in said subject, optionally in the absence of comparison to another subject. These methods may be practiced repeatedly, with a variety of amounts or concentrations of the compound to determine the level of inhibition over a range of conditions. The methods may also be used to determine that the level of inhibition is undetectable.

An exemplary method of determining the level of inhibition of a target binding compound may comprise
a) administering a target binding inhibitor compound to a subject;
b) determining the level of inhibitory activity or efficacy against a disease or unwanted condition as disclosed herein in comparison to a subject (or group of subjects) that has not been administered said compound or that has been administered a different amount of said compound or administered said compound under different administration protocols (such as, but not limited to, frequency of administration or amount of compound administered).

The comparison may also be made between different target binding compounds to determine their relative levels of activity. The subjects are animals, preferably human, and may be those that are part of a clinical or pre-clinical trial or test of one or more target binding compound. The determination of the level of inhibitory activity may also be performed outside, or after, a clinical trial to identify the level of inhibition by a target binding inhibitor compound and can be made in a variety of ways as would be known to the skilled practitioner for a disease or unwanted condition.

Other features and advantages of the invention will be apparent from the following detailed description and claims.

### BRIEF DESCRIPTION OF THE DRAWINGS

FIG. 1 depicts BIRB 796, imatinib mesylate, and BAY 43-9006.

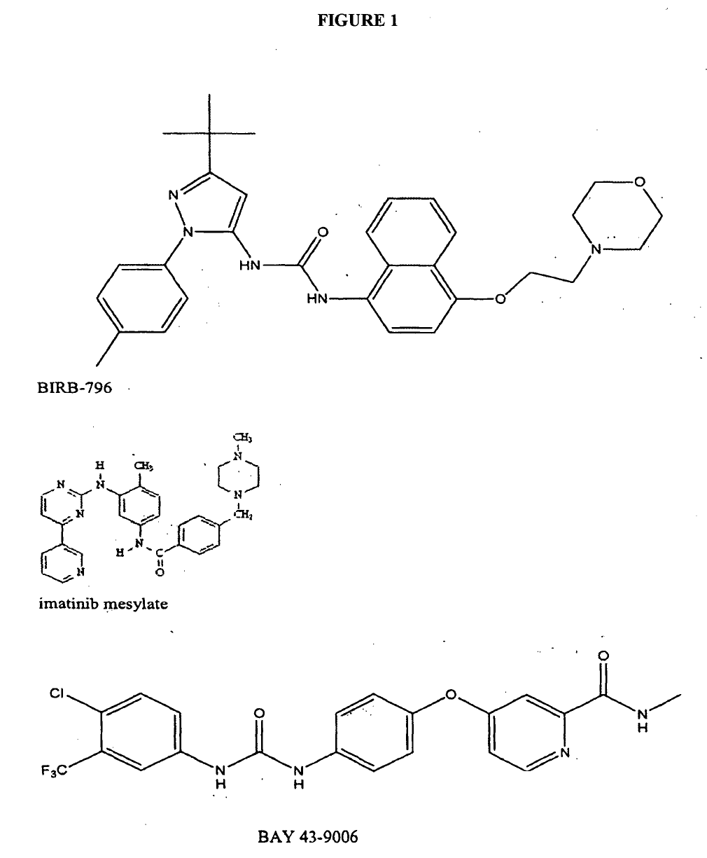

### DETAILED DESCRIPTION OF THE INVENTION

### Definitions

As used herein, the terms “LCK kinase”, “p38/MAPK14”, “Abl kinases”, “PDGFR”, “VEGFR2” and “target” protein or polypeptide includes analogs of these protein kinases which may be obtainable from other animals or humans with deviations in amino acid sequences or encoding nucleotide sequences relative to known sequences encoding these kinases. The term “analog” refers to a molecule which is structurally similar or has the same function or activity as any of the above kinases. As a non-limiting example, an analog of the LCK protein kinase can be specifically bound by an antibody or T cell that specifically binds to LCK protein kinase. Naturally occurring analogs from other animals and other humans, as well as alleles thereof (including those resulting from genetic polymorphisms), may be used in the practice of the invention. Synthetic analogs resulting from genetic engineering, such as those based upon the use of conservative amino acid substitutions or degeneracy in the genetic code, may also be used.

The term “homolog” refers to a molecule which exhibits homology to another molecule, by for example, having sequences of chemical residues that are the same or similar at corresponding positions. Homologs of a target protein may be used in the practice of the invention, especially in certain methods as disclosed herein.

As used herein, the term “polynucleotide” means a polymeric form of nucleotides, preferably of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T) (as shown for example in SEQ ID NO: 1) can also be uracil (U); this definition pertains to the differences between the chemical structures of DNA and RNA, in particular the observation that one of the four major bases in RNA is uracil (U) instead of thymidine (T).

As used herein, a target protein's genes and proteins include the known human genes and proteins thereof, as well as structurally and/or functionally similar analogs thereof. Analogs of a target protein generally share at least about 50%, 60%, 70%, 80%, 90% or more amino acid homology (using BLAST criteria) to known amino acid sequences of said protein. Nucleotide analogs of a target protein preferably share 50%, 60%, 70%, 80%, 90% or more nucleic acid homology (using BLAST criteria) to known nucleic acid sequences encoding said protein.

The target proteins of the invention include those specifically identified herein, as well as allelic variants, conservative substitution variants, analogs and homologs that can be isolated/generated and characterized without undue experimentation following the methods outlined herein or readily available in the art. Fusion proteins that combine parts of different target proteins or fragments thereof, as well as fusion proteins of a target protein and a heterologous polypeptide are also included and may be used in the practice of the invention.

In general, naturally occurring allelic variants of human LCK kinase, p38/MAPK14, imatinib mesylate resistant and sensitive Abl kinases, PDGFR, or VEGFR2 share a high degree of structural identity and homology (e.g., 90% or more homology). Typically, allelic variants of a target protein contain conservative amino acid substitutions. Conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15, or more conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. pages 13-15 “Biochemistry” 2^{nd} ED. Lubert Stryer ed (Stanford University); Henikoff et al., PNAS 1992 Vol 89 10915-10919; Lei et al., J Biol Chem 1995 May 19; 270(20):11882-6).

Analogs of a target protein can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the variant DNA.

As defined herein, an analog of a target protein has the distinguishing attribute of having at least one epitope that is “cross reactive” with a target protein, respectively, as known in the field. The term “cross reactive” means that an antibody or T cell that specifically binds to a target protein analog also specifically binds to at least one corresponding known target protein. A polypeptide ceases to be an analog when it no longer contains any epitope capable of being recognized by an antibody or T cell that specifically binds to a target protein. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9):865-73; Schwartz et al., J Immunol (1985) 135(4):2598-608.

Target polypeptides may be generated using standard peptide synthesis technology or using chemical cleavage methods well known in the art. Alternatively, recombinant methods can be used to generate nucleic acid molecules that encode a target polypeptide. In one embodiment, nucleic acid molecules provide a means to generate defined fragments of a target protein or analog thereof. The polypeptides may contain covalent modifications and still be used in the practice of the invention. Non-limiting examples of such modifications include reacting the amino acid residues of a target polypeptide with an organic derivatizing agent that is capable of reacting with selected side chains or the N- or C-terminal residues of a target protein; altering the native glycosylation pattern of the target protein; and linking the target polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The target proteins of the present invention can also be modified to form a chimeric molecule comprising a target protein fused to another polypeptide or amino acid sequence. Such a chimeric molecule can be synthesized chemically or recombinantly and used in the practice of the invention. A chimeric molecule can comprise a fusion of a target protein with a polyhistidine epitope tag, which provides an epitope to which immobilized nickel can selectively bind, with cytokines or with growth factors. In an alternative embodiment, the chimeric molecule can comprise a fusion of a target protein with an immunoglobulin or a particular region of an immunoglobulin. For the production of immunoglobulin fusions see, e.g., U.S. Pat. No. 5,428,130 issued Jun. 27, 1995. Alternatively, the fusion can be with a signaling moiety, such as a fluorescent protein or chromophore, including, but not limited to green fluorescent protein.

A target polypeptide may also be expressed as a fusion with a phage coat protein for expression on the surface of phage particles. This approach is described in copending U.S. patent application Ser. Nos. 10/115,442, filed 2 Apr. 2002, and application Ser. No. 10/406,797 filed on 2 Apr. 2003 (or PCT International Application PCT/US03/10247 filed 2 Apr. 2003), both of which are incorporated by reference as if fully set forth. The expressed proteins may be used with a compound that binds a target protein, such as BIRB 796, imatinib mesylate, and/or BAY 43-9006, in an immobilized form as described in these applications for use in screens for other compounds that bind the target protein.

### Methods of Treatment

Novel protein targets for BIRB 796, imatinib mesylate, and BAY 43-9006 are described herein. Based on these novel interactions, methods of treatment of disease conditions are provided.

In one embodiment of the invention, a method of treating imatinib mesylate resistant chronic myelogenous leukemia (CML) is provided by contacting a imatinib mesylate resistant Abl polypeptide with a compound that binds p38/MAPK14; preferably the compound is BIRB-796. Similarly, the invention provides a method of treating inflammation or inducing immunosuppression by contacting a LCK protein kinase polypeptide with a compound that binds Bcr-Abl, c-kit, and/or PDGFR; preferably, the compound is imatinib mesylate.

In other embodiments, a method of treating inflammation, psoriasis, or rheumatoid arthritis by contacting a p38/MAPK14 polypeptide with a compound that binds Raf kinase, imatinib mesylate resistant or sensitive Abl kinase, PDGFR, and/or VEGFR2; a method of treating angiogenesis or neovasculature by contacting a PDGFR or VEGFR2 polypeptide with a compound that binds Raf kinase, p38/MAPK14, and/or imatinib mesylate resistant or sensitive Abl kinase; and a method of treating smooth cell proliferation, intimal hyperplasia, or restenosis by contacting a VEGFR2 polypeptide with a compound that binds Raf kinase; p38/MAPK14, imatinib mesylate resistant or sensitive Abl kinase, and/or PDGFR are provided. Preferably, these methods are practiced with BAY 43-9006.

In further embodiments, the invention provides methods of treating and/or preventing a condition selected from chronic myelogenous leukemia (CML), imatinib mesylate resistant chronic myelogenous leukemia (CML), inflammation, immunosuppression, psoriasis, rheumatoid arthritis or Crohn's disease, smooth cell proliferation, intimal hyperplasia, restenosis, and/or angiogenesis or neovasculature (particularly in association with solid tumors, metastasized tumors, osteosarcoma, small cell lung cancer, angiomyolipoma, neoplasms associated with tuberous sclerosis, or myeloproliferative disease; or diabetic retinopathy (DR), ocular neovasculature, or macular degeneration) by administering an effective amount of a compound identified by a method of the invention as provided herein to treat or prevent said condition in a subject. In other embodiments, BAY 43-9006 are used in the treatment and/or prevention of these disorders.

More particularly, BAY 43-9006 may be used in treating and/or preventing a condition selected from cancer (especially solid tumors, metastasized tumors, osteosarcoma, small cell lung cancer, CML, or angiomyolipoma); diabetic retinopathy (DR); ocular neovasculature; and macular degeneration in a subject identified as in need of such treatment and/or prevention by administering an effective amount of BAY 43-9006 to said subject.

Uses provided by the present invention also include a method of lowering chronic myelogenous leukemia (CML) related Abl kinase activity, p38/MAPK14 kinase activity, PDGFR and/or VEGFR2 protein kinase activity in a subject identified as in need thereof by administering an effective amount of BAY 43-9006 to said subject. Another use is a method of lowering LCK protein kinase activity in a subject identified as in need thereof by administering an effective amount of imatinib mesylate to said subject.

The invention also provides for the use of BAY 43-9006 to produce actions that result from the targeting or inhibition of platelet-derived growth factor (PDGF) α- and/or β-receptor mediated protein kinase activity.

In a preferred embodiment, BIRB-796 is used in the treatment of Gleevec resistant leukemia. BIRB-796 can be used in the treatment and/or prevention of Gleevec resistant leukeamia caused by mutations of the Abl tyrosine kinase. In particular, BIRB-796 can be used in the treatment and/or prevention of Gleevec resistant leukemia caused by Thr334Ile mutant of Abl tyrosine kinase.

In another preferred embodiment, Gleevec is used in the treatment of LCK-mediated diseases. Such LCK mediated diseases include, but are not limited to, inflammation and autoimmune diseases. Also, Gleevec can be used in the treatment and/or prevention of organ rejection in transplant patients. Further, Gleevec can be used to produce immunosuppression in patients in need thereof, such as transplant patients and patients suffering from autoimmune disorders and/or inflammatory disorders.

BIRB-796 can be used in the treatment of diseases mediated by EPHA2, EPHA3, EPHA4, EPHA5, EPHA6, EPHA7, EPHA8, EPHB1, FRK, MAPK9/JNK2, MAPK10/JNK3, LCK, MKNK2, NTRK1, MAPK11/p38-beta, MAPK12/p38-gamma, or a combination thereof. In addition to the treatment of LCK mediated diseases, Gleevec can be used in the treatment of diseases mediated by MAPK8/JNK1, MAPK9/JNK2, MAPK10/JNK3, PDGFRB, or a combination thereof. Additional diseases that can be treated with BAY 43-9006 include ABL kinase mediated diseases such as those mediated by ABL1, ABL1(E274K), ABL1(H415P), ABL1(M370T), ABL1(Q271H), ABL1(T334I), ABL1(Y272F), ABL2, or combination thereof. Diseases mediated by MAPK11/p38-beta and/or MAPK12/p38-gamma may be also be treated with BAY 43-9006.

The term “kinase-mediated” disease and such other references to diseases/disorders mediated by kinases referred to herein is intended to encompass diseases in which directly or indirectly modulating the activity and/or production of the kinase is desirable. This modulation can be either upstream or downstream of the signaling cascades of the kinases. The compounds discussed herein can be used to modulate several kinases. This modulation can include reducing, increasing, or stabilizing the activity of the kinases.

A target protein binding compound of the invention, such as BIRB 796, imatinib mesylate, and/or BAY 43-9006, may be administered to a subject upon determination of the subject as having a disease or unwanted condition that would benefit by treatment with said compound. The determination may be made by medical or clinical personnel as part of a diagnosis of a disease or condition in a subject. Preferred embodiments include methods for the use of a target protein binding compound to provide the effects of BIRB 796, imatinib mesylate, and/or BAY 43-9006 after administration to a subject. Exemplary effects include, but are not limited to, the treatment of cancer, including, but not limited to leukemia and cancers in which an inhibition of p38/MAPK14, Abl kinase, PDGFR, and/or VEGFR2 would be beneficial; the treatment of inflammation or the generation of immunosuppression, especially that resulting from the inhibition of LCK kinase; and the treatment of angiogenesis, neovasculature, and vascular permeability, especially that associated with cancer and ocular diseases. The binding compound may also be used in the prevention of such conditions, which may be viewed as reducing the probability of a subject having one or more of the conditions. In one embodiment of the invention, BAY 43-9006 may be used to simultaneously target PDGFR and VEGFR2 to inhibit the proliferation of tumor vasculature.

The methods of the invention may comprise the administration of a target protein binding compound alone or in combination with one or more other molecule or other agents suitable for treatment of a disease or unwanted condition as disclosed herein. The target protein binding compound is preferably administered in an effective amount such that the disease or unwanted condition is alleviated relative to the absence of the compound's use in a subject. The subject is preferably human, and repeated administration over time is within the scope of the present invention.

In some embodiments, the methods of the invention involve contacting a cell with a target protein binding compound that inhibits one or more of the activities of the target protein activity associated with the cell. These methods can be performed in vitro (e.g., by culturing the cell with the agent) or, alternatively, in vivo (e.g, by administering the agent to a subject). They may also be performed ex vivo, as in the case of cells obtained from a subject and treated in vitro followed by their return to the subject.

The identification of the additional targets of the invention for treating diseases and unwanted conditions permits a number of therapeutic approaches to the treatment thereof. Accordingly, therapeutic approaches that inhibit the function and activity of a target protein are provided by the invention. These therapeutic approaches generally fall into two classes. One class comprises various methods for affecting the binding or association of a target protein with its binding partner or with other proteins. Another class comprises a variety of methods for inhibiting the transcription of the target protein gene or translation of target protein mRNA.

In one preferred embodiment of the invention, a small molecule identified as binding a target protein may be used to inhibit its function or activity. Alternatively, a target protein may be targeted by antibody-based therapeutic strategies. A number of antibody strategies are known in the art for targeting intracellular molecules, including the intracellular expression of single chain antibodies. Antibodies can be introduced into a patient such that the antibody binds to a target protein and inhibits a function, such as an interaction with a binding partner. Alternatively, the antibody affects ligand binding or signal transduction pathways mediated by a target protein.

The present invention also comprises various methods and compositions for inhibiting the transcription of target protein encoding sequences. Similarly, the invention also provides methods and compositions for inhibiting the translation of target protein mRNA into protein.

In vivo, the effect of a target protein inhibiting therapeutic composition can be evaluated in a suitable animal model. In vivo assays that evaluate the inhibition of target protein function or activity are also useful in evaluating therapeutic compositions.

The inhibitors of the invention may also be used in prophylactic methods to prevent in a subject, a disease or unwanted condition associated with target protein expression or activity, by administering to the subject an agent which affects target protein expression or at least one target protein activity. Subjects at risk for a disease which is caused or contributed to by aberrant target protein expression or activity can be identified by any appropriate prognostic assays as known in the field. Administration of a prophylactic agent can occur prior to the manifestation of symptoms characteristic of aberrant target protein levels, such that a disease or condition is prevented or, alternatively, delayed in its progression.

As used herein, an effective amount of a compound or agent refers to an amount sufficient to achieve its intended purpose. Determination of the effective amounts is well within the capability of those skilled in the art based upon the achievement of a desired effect. An effective amount will depend on factors including, but not limited to, the size of a subject and/or the degree to which the disease or unwanted condition from which a subject suffers has progressed. The effective amount will also depend on whether the compound or agent is administered to the subject in a single dosage or periodically over time.

The present invention provides methods, pharmaceutical compositions, and kits for the treatment of subjects. As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like.

The term “treating” and its grammatical equivalents as used herein include achieving a therapeutic benefit and/or a prophylactic benefit. By therapeutic benefit is meant eradication or amelioration of the underlying disorder being treated. For example, in a cancer patient, therapeutic benefit includes eradication or amelioration of the underlying cancer. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder.

For prophylactic benefit, a composition of the invention may be administered to a patient at risk of developing a kinase-mediated condition, or to a patient reporting one or more of the physiological symptoms of such conditions, even though a diagnosis of the condition may not have been made.

### Compositions and Formulations

The target protein binding compounds of the invention are preferably used to prepare a medicament, such as by formulation into pharmaceutical compositions for administration to a subject using techniques generally known in the art. A summary of such pharmaceutical compositions may be found, for example, in Remington's Pharmaceutical Sciences, Mack Publishing Co., Easton, Pa. The compounds of the invention can be used singly or as components of mixtures. Preferred forms of the compounds are those for systemic administration as well as those for topical or transdermal administration. Formulations designed for timed release are also with the scope of the invention.

If necessary or desirable, compounds of the invention may be administered in combination with other therapeutic agents. The choice of therapeutic agents that can be co-administered with the compositions of the invention will depend, in part, on the condition being treated.

The modulators may be administered per se or in the form of a pharmaceutical composition wherein the active compound(s) is in an admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries, which facilitate processing of the active compounds into preparations that can be used pharmaceutically. Proper formulation is dependent upon the route of administration chosen. The modulators useful in the present invention can be delivered to the patient using a number of routes or modes of administration, including oral, buccal, topical, rectal, transdermal, transmucosal, subcutaneous, intravenous, and intramuscular applications, as well as by inhalation.

Methods for the preparation of compositions comprising the compounds of the invention include formulating the derivatives with one or more inert, pharmaceutically acceptable carriers to form either a solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein.

Compounds of this invention may also be integrated into foodstuffs, e.g., cream cheese, butter, salad dressing, or ice cream to facilitate solubilization, administration, and/or compliance in certain patient populations.

The compounds of the invention may be labeled isotopically (e.g. with a radioisotope) or by other means, including, but not limited to, the use of chromophores or fluorescent moieties, bioluminescent labels, or chemiluminescent labels. The compositions may be in conventional forms, either as liquid solutions or suspensions, solid forms suitable for solution or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. Of course, these compositions may also contain minor amounts of nontoxic, auxiliary substances, such as wetting or emulsifying agents, pH buffering agents, and so forth.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Aqueous suspensions may contain a compound of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

For injection, the compounds of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some embodiments, pharmaceutical compositions comprising compounds of the present invention exert local and regional anti-inflammatory effects when administered topically or injected at or near particular sites of inflammation. For example, ocular allergic, inflammatory and/or autoimmune conditions can be effectively treated with ophthalmic solutions, suspensions, ointments or inserts comprising one or more compounds of the present invention. Allergic, inflammatory and/or autoimmune conditions of the ear can be effectively treated with otic solutions, suspensions, ointments or inserts comprising one or more compounds of the present invention. Allergic, inflammatory and/or autoimmune conditions of the skin and skin structures can be effectively treated with skin ointments comprising one or more compounds of the present invention in an oleaginous hydrocarbon base, an anhydrous absorption base, a water-in-oil absorption base, an oil-in-water water-removable base and/or a water-soluble base. Gastrointestinal allergic, inflammatory and/or autoimmune conditions can be effectively treated with orally- or rectally delivered solutions, suspensions, ointments, enemas and/or suppositories comprising one or more compounds of the present invention. Respiratory allergic, inflammatory and/or autoimmune conditions can be effectively treated with aerosol solutions, suspensions or dry powders comprising one or more compounds of the present invention.

For example, for treating inflammatory and/or autoimmune conditions, a cream comprising a compound of the invention may be topically applied to the affected site, for example, sites displaying red plaques or dry scales in psoriasis, or areas of irritation and dryness in dermatitis. As another example, for treating inflammatory bowel disease, a suppository formulation of a compound disclosed herein can be used. In such embodiments, the active ingredient produces a benefit locally at or near the site of application, rather than systemically.

Direct topical application, e.g., of a viscous liquid, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983.

In some preferred embodiments, the compounds of the present invention are delivered in soluble rather than suspension form, which allows for more rapid and quantitative absorption to the sites of action. In general, formulations such as jellies, creams, lotions, suppositories and ointments can provide an area with more extended exposure to the compounds of the present invention, while formulations in solution, e.g., sprays, provide more immediate, short-term exposure.

The formulations also may comprise suitable solid or gel phase carriers or excipients that increase penetration or help delivery of inhibitory compounds of this invention across the permeability barrier of the skin. Many of these penetration-enhancing compounds are known in the art of topical formulation. Examples of such carriers and excipients include humectants (e.g., urea), glycols (e.g., propylene glycol and polyethylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, ORGELASE, calcium carbonate, calcium phosphate, various sugars, starches, cellulose derivatives, gelatin, other polymers and water. In some embodiments, the pharmaceutical compositions will include one or more penetration enhancers such as water, methanol, ethanol, 2-propanol, dimethyl sulfoxide, decylmethyl sulfoxide, tetradecylmethyl sulfoxide, 2-pyrrolidone, N-methyl-2-pyrrolidone, N-(2-hydroxyethyl)pyrrolidone, laurocapram, acetone, dimethylacetamide, dimethylformamide, tetrahydrofurfuryl alcohol, L-α-amino acids, anionic surfactants, cationic surfactants, amphoteric surfactants, nonionic surfactants, fatty acids, fatty alcohols, clofibric acid amides, hexamethylene lauramide, proteolytic enzymes, α-bisabolol, d-limonene, urea, N,N-diethyl-m-toluamide, and the like.

In some embodiments, the pharmaceutical compositions will include one or more antimicrobial preservatives such as quaternary ammonium compounds, organic mercurials, p-hydroxy benzoates, aromatic alcohols, chlorobutanol, and the like.

Pharmaceutical compositions suitable for use in the present invention include compositions wherein the active ingredients are present in an effective amount, i.e., in an amount effective to achieve therapeutic and/or prophylactic benefit in a condition being treated. The actual amount effective for a particular application will depend on the condition or conditions being treated, the condition of the subject, the formulation, and the route of administration, as well as other factors known to those of skill in the art. Determination of an effective amount of the compounds of the present invention is well within the capabilities of those skilled in the art, in light of the disclosure herein, and will be determined using routine optimization techniques.

In therapeutic use, the compounds of the invention are administered to a subject at dosage levels of from about 0.05 mg/kg to about 10.0 mg/kg of body weight per day. For a human subject of approximately 70 kg, a dosage of from about 40 mg to about 600 mg per day may be used as a non-limiting example. Preferred doses include about 1 mg/kg, about 2.5 mg/kg, about 5 mg/kg, and about 7.5 mg/kg. Lower or higher doses than those disclosed herein may be used, as required. Such dosages, however, may be altered depending on a number of variables, not limited to the activity of the compound used, the condition to be treated, the mode of administration, the requirements of the individual subject, the severity of the condition being treated, and the judgment of the practitioner. The foregoing ranges are merely suggestive, as the number of variables in regard to an individual treatment regime is large, and considerable excursions from these recommended values are not uncommon.

The effective amount for use in humans can be determined from animal models. For example, a dose for humans can be formulated to achieve circulating, liver, topical and/or gastrointestinal concentrations that have been found to be effective in animals.

The effective amount when referring to an inhibitor of the invention will generally mean the dose ranges, modes of administration, formulations, etc., that have been recommended or approved by any of the various regulatory or advisory organizations in the medical or pharmaceutical arts (eg, FDA, AMA) or by the manufacturer or supplier. In some embodiments, administration of compounds of the present invention may be intermittent, for example administration once every two days, every three days, every five days, once a week, once or twice a month, and the like. In some embodiments, the amount, forms, and/or amounts of the different forms may be varied at different times of administration.

The methods of the present invention can be practiced with the compounds disclosed herein and also with analogs and derivatives thereof. Also, prodrugs and active metabolites of the compounds of the present invention may be used. The compounds of the present invention may exhibit the phenomena of tautomerism, conformational isomerism, geometric isomerism, and/or optical isomerism. The invention covers any tautomeric, conformational isomeric, optical isomeric and/or geometric isomeric forms of the compounds, as well as mixtures of these various different forms. Other suitable derivatives of Gleevec are disclosed in U.S. Pat. No. 5,521,184, which is hereby incorporated by reference in its entirety.

### Uses and Methods of the Invention

The target polypeptides, as well as fragments, homologs, and analogs thereof, of the invention have a number of different specific uses. In particular, they may be used to identify additional compounds that bind LCK kinase, p38/MAPK14, imatinib mesylate resistant and sensitive Abl kinases, PDGFR, and/or VEGFR2, including additional compounds that inhibit their function or activity. In one preferred embodiment, a target polypeptide is expressed as a fusion protein for expression on phage particles which may then be screened against a library of compounds, either in solution or in immobilized form.

The invention also provides for the use of polynucleotides/nucleic acid molecules, proteins, protein analogs, and target binding compounds described herein in one or more of the following methods: a) expression of target polypeptides; b) screening assays; c) methods of determining effects of a compound on one or more target; and d) methods of treatment (e.g., therapeutic and prophylactic). The polynucleotides of the invention can be used, for example, to express a target protein (e.g., via a recombinant expression vector in a host cell), to detect target encoding mRNA (e.g., in a biological sample) or a genetic alteration in a target gene. The target proteins can be used to identify additional molecules that bind and/or inhibit the activity of one or more targets. The identified molecules may be used to treat diseases or unwanted conditions characterized by undesirable levels of target protein production or of target protein activity.

The invention thus includes methods to screen for drugs or compounds which bind and/or modulate a target protein's activity, which drugs or compounds may be used to treat disorders requiring a decrease in the function or activity of one or more target proteins. The methods include a method for identifying compounds, i.e., candidate or test compounds or agents (such as, but not limited to, peptides; peptidomimetics; small molecules of less than 5000, less than 4500, less than 4000, less than 3500, less than 3000, less than 2500, less than 2000, less than 1500, less than 1000, or less than 500 Daltons; or other drugs) which bind to a target protein and optionally have an inhibitory effect thereon, or have an inhibitory effect on the expression of a target protein. Preferred are compounds that bind with a K_{d} of less than about 500 μM, less than about 100 μM, less than about 50 μM, less than about 10 μM, less than about 5 μM, less than about 1 μM, less than about 0.5 μM, or less than about 0.1 μM.

The invention thus provides a method for identifying a compound as binding a target polypeptide by contacting the polypeptide, or a phage particle expressing the polypeptide on its surface, with a test compound, and determining whether the polypeptide binds to the test compound. The binding of the test compound to the polypeptide may be detected by direct detection of interactions between the test compound and the polypeptide; detection of binding by indirect detection of interactions between the test compound and the polypeptide; detection of binding using a competition binding assay; and detection of binding using an assay for the polypeptide's activity. In another embodiment, a method for identifying a compound which inhibits the activity of a target polypeptide is provided by contacting a target polypeptide with a test compound and determining the extent to which the test compound inhibits the activity of the polypeptide. The methods may be performed in vitro or in vivo, such as in cells from an animal (including cell lines) or cells in an animal.

Other non-limiting examples of binding assays include BIACORE-type binding assays, DiscoveRx type binding assays; fluorescence and fluorescence polarization; FRET (fluorescence energy transfer); fluorescence enhancement/quenching; effects on protein stability (binding stabilizes the protein, affecting unfolding thermodynamics as measured by a melting temperature, or the concentration of denaturants required to unfold the protein); general migration, rotation properties of the protein or small molecule; interference with chemical modification (e.g. if there is a reactive group at an active site which can be chemically labeled, this may be blocked if a small molecule binds at the active site); NMR-based measurements; crystallographic methods; other indirect cell-based methods (or methods based on artificial cells, micelles etc.); and 3-hybrid type methods.

In one embodiment of the invention, a cell-free assay is provided in which a target protein or biologically active portion thereof is contacted with a test compound and the ability of the test compound to bind to the protein or biologically active portion thereof is determined. Preferably, the compound is a small molecule as described herein. In a preferred embodiment, the assay includes contacting the target protein or biologically active portion thereof with a known compound which binds the target to form an assay mixture, contacting the assay mixture with a test compound, and determining the ability of the test compound to interact with a target protein, wherein determining the ability of the test compound to interact with a target protein comprises determining the ability of the test compound to preferentially bind to the target or biologically active portion thereof as compared to the known compound.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). The methods may also be used to confirm the binding of a compound to a target protein or to confirm the effect of a compound on a target's function or activity.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew Chem. Int. Ed. Engl. 33:2061; and in Gallop et al. (1994) J. Med. Chem. 37:1233.

Libraries of compounds maybe presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991)J. Mol. Biol. 222:301-310); (Ladner supra.).

One can screen peptide libraries to identify molecules that interact with a target protein's sequences. Alternatively, the particles are screened against small molecules of interest as described herein. Conversely, target polypeptides may be expressed on bacteriophage particles and then screened against peptides or small molecules in solution or immobilized form. Accordingly, peptides and small molecules that bind and inhibit a target protein are identified without any prior information on the structure of the peptides and small molecules.

In another embodiment, an assay is a cell-based assay comprising contacting a cell expressing a target binding ligand molecule with a test compound and determining the ability of the test compound to affect the binding of the target to the ligand. Determining the ability of the test compound to increase or decrease the binding of a target ligand can be accomplished, for example, by determining the ability of the target protein to interact with the ligand, such as by determination of direct binding between the target and a ligand thereof, such as by coupling the target protein with a radioisotope, fluorescent, or enzymatic label such that binding of the protein to a ligand molecule can be determined by detecting the labeled protein in a complex.

Alternatively, cell lines that express a target protein are used to identify protein-protein interactions mediated by a target protein using immunoprecipitation techniques (see, e.g., Hamilton B J, et al. Biochem. Biophys. Res. Commun. 1999, 261:646-51). The target proteins can also be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins or factors, which bind to or interact with a target protein. The identified proteins or factors may be used as a ligand molecule which binds a target protein as described herein. The invention also provides for determining the ability of a compound to affect the binding of a target protein to a ligand molecule, without labeling either of the binding members. For example, a microphysiometer can be used to detect the interaction of with its ligand without the labeling of either the target protein or the ligand. McConnell, H. M. et al. (1992) Science 257:1906-1912.

In another embodiment, determining the ability of a target protein to bind to or interact with a ligand molecule can be accomplished by detecting the activity of the ligand.

In more than one embodiment of the above assay methods of the present invention, it may be desirable to immobilize either a target protein or its ligand molecule to facilitate separation of complexed from uncomplexed forms of one or both of the proteins, as well as to accommodate automation of the assay. Binding of a test compound to a target protein, or interaction of a target protein with a ligand molecule in the presence and absence of a candidate compound, can be accomplished in any vessel suitable for containing the binding members and other reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/kinase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound and either the non-adsorbed ligand or target protein, respectively, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of target protein binding or activity determined using standard techniques.

Other techniques for immobilizing proteins on matrices can also be used in the screening assays of the invention. For example, either a target protein or a ligand molecule thereof can be immobilized utilizing conjugation of biotin and streptavidin. Biotinylated target protein molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a target protein or ligand molecules but which do not interfere with binding of the target protein to its ligand can be derivatized to the wells of the plate, and unbound target or target protein trapped in the wells by antibody conjugation. Methods for detecting such complexes, in addition to those described above for the GST-immobilized complexes, include immunodetection of complexes using antibodies reactive with the target protein or ligand, as well as enzyme-linked assays which rely on detecting an enzymatic activity associated with the target protein or ligand.

This invention further pertains to novel compounds and agents identified by the above-described screening assays. Accordingly, it is within the scope of this invention to further use an agent identified as described herein in an appropriate animal subject, such as a human. For example, a target binding compound identified as described herein can be used in an animal model to determine the efficacy, toxicity, or side effects of treatment with such a compound. Alternatively, an agent identified as described herein can be used in an animal subject to provide additional information concerning the mechanism of action of such an agent. Furthermore, this invention pertains to uses of novel agents identified by the above-described screening assays for treatments as described herein.

With respect to screening assays, the invention provides for monitoring the influence of agents (e.g., drugs or compounds) on the level of expression or activity of a target protein, such as in pre-clinical or clinical trials or in post-trial use. For example, the level of effectiveness of BIRB 796, imatinib mesylate, BAY 43-9006, or an agent determined by a screening assay to decrease target gene expression, protein levels, or downregulate a target protein activity, can be monitored in clinical trials of subjects exhibiting undesirable target gene expression, protein levels, or upregulated activity. In such pre-clinical or clinical trials or post-trial uses, the expression or activity of a target protein gene, and preferably, other genes that have been implicated in a disorder, can be used as a “read out” or markers of the phenotype of a particular cell. The levels of gene expression (i.e., a gene expression pattern) can be quantified by Northern blot analysis; the use of DNA chips or microarrays or bead mediated arrays (like those of Illumina, Inc.); RT-PCR; or other techniques known in the art. Alternatively, expression can be determined by measuring the amount of protein produced, by one of the methods as described herein, or by measuring the levels of activity of a target protein. In this way, the gene expression pattern can serve as a marker, indicative of the physiological response of the cells to the agent. Accordingly, this response state may be determined before, and at various points during treatment of the individual with the agent or after administration of the agent to the individual.

In a preferred embodiment, the present invention provides a method for monitoring the effectiveness of treatment of a subject by administration of a compound that binds a target protein comprising the steps of (i) obtaining a pre-administration sample from a subject prior to administration of the agent; (ii) detecting the level of expression of a target protein, mRNA, or genomic DNA in the pre-administration sample; (iii) obtaining one or more post-administration samples from the subject; (iv) detecting the level of expression or activity of the target protein, mRNA, or genomic DNA in the post-administration samples; (v) comparing the level of expression or activity of the target protein, mRNA, or genomic DNA in the pre-administration sample with the target protein, mRNA, or genomic DNA in the post administration sample or samples; and optionally (vi) altering the administration of the agent to the subject accordingly. For example, increased administration of an inhibitory compound may be desirable to increase the inhibition of a target protein to higher levels than detected, i.e., to increase the effectiveness of the compound. According to such an embodiment, target protein expression or activity may be used as an indicator of the effectiveness of an agent, even in the absence of an observable phenotypic response.

In other embodiments, the invention provides a method for identifying a compound for the treatment of chronic myelogenous leukemia (CML) comprising contacting an Abl polypeptide, or a fragment or portion thereof, or a phage particle expressing the polypeptide or fragment or portion on its surface, or a cell expressing the polypeptide or fragment or portion, with a test compound that binds Raf kinase, p38/MAPK14, PDGFR, and/or VEGFR2; and determining whether the polypeptide or a fragment or portion thereof, binds to the test compound.

The invention also provides analogous methods for identifying a compound for the treatment of imatinib mesylate resistant chronic myelogenous leukemia (CML) by contacting a imatinib mesylate resistant Abl polypeptide (or other formats as described above for an Abl polypeptide) with a test compound that binds p38/MAPK14; and determining whether the polypeptide or a fragment or portion thereof, binds to the test compound. The same formats can be used to identify a compound for the treatment of inflammation or inducing immunosuppression by contacting a LCK protein kinase polypeptide, or a fragment or portion thereof, with a test compound that binds Bcr-Abl, c-kit, and/or PDGFR; and determining whether the polypeptide or a fragment or portion thereof, binds to the test compound. The formats can also be used for identifying a compound for the treatment of inflammation, psoriasis, rheumatoid arthritis or Crohn's disease by contacting a p38/MAPK14 polypeptide, or a fragment or portion thereof, with a test compound that binds Raf kinase, imatinib mesylate resistant or sensitive Abl kinase, PDGFR, and/or VEGFR2, and determining whether the polypeptide or a fragment or portion thereof, binds to the test compound.

In other embodiments, the formats may be used for identifying a compound for the treatment of angiogenesis or neovasculature by contacting a PDGFR or VEGFR2 polypeptide, or a fragment or portion thereof, with a test compound that binds Raf kinase, p38/MAPK14, and/or imatinib mesylate resistant or sensitive Abl kinase, and determining whether the polypeptide or a fragment or portion thereof, binds to the test compound. Similarly, the formats are used for identifying a compound for the treatment of smooth cell proliferation, intimal hyperplasia, or restenosis by contacting a VEGFR2 polypeptide, or a fragment or portion thereof, with a test compound that binds Raf kinase, p38/MAPK14, imatinib mesylate resistant or sensitive Abl kinase, and/or PDGFR, and determining whether the polypeptide or a fragment or portion thereof, binds to the test compound.

A compound identified by any method of the invention may be formulated as a pharmaceutical composition comprising the compound and a pharmaceutically acceptable excipient.

Having now generally described the invention, the same will be more readily understood through reference to the following examples which are provided by way of illustration, and are not intended to be limiting of the present invention, unless specified.

### EXAMPLES

Identification of Interactions Between Molecules and Target Proteins

BIRB 796, imatinib mesylate, and BAY 43-9006 were screened for target protein binding activity in a standard phage-based competition binding assay as described in copending U.S. patent application Ser. No. 10/115,442, filed 2 Apr. 2002, and Ser. No. 10/406,797 filed on 2 Apr. 2003 (or PCT International Application PCT/US03/10247 filed 2 Apr. 2003).

60 kinase proteins were displayed on phage particle surfaces and assayed for their binding to BIRB 796. The binding constants (micromolar) are shown in Table 1, wherein ABL1 refers to human Abl tyrosine kinase; ABL1 (T334I) refers to the Thr334Ile (also known as Thr315Ile) mutant of human Abl tyrosine kinase; EPHA3 (Nuk) is a human tyrosine protein kinase receptor; FRK refers to human Fyn-related kinase (tyrosine protein kinase); JNK2A2/MAPK9 refers to the human MAPK9 protein kinase (aka mitogen-activated protein kinase 9 or c-Jun kinase 2); JNK3A1/MAPK10 refers to the human MAPK10 protein kinase; LCK and p38/MAPK14 are the human forms thereof and are described herein.

TABLE 1

| Gene Symbol | BIRB-796 |
|---|---|
| ABL1 | 0.69 |
| ABL1(T334I) | 0.020 +/− 0.008 |
| EPHA3 | 0.7 |
| FRK | 0.27 |
| JNK2A2/MAPK9 | 0.002 |
| JNK3A1/MAPK10 | 0.06 |
| LCK | 0.26 |
| p38/MAPK14 | <0.001 |

48 kinase proteins were displayed on phage particle surfaces and assayed for their binding to imatinib mesylate (Gleevec). The binding constants (micromolar) are shown in Table 2, with the identifiers are as described above except PDGFRb refers to the human form of platelet-derived growth factor (PDGF) receptor, β form and is described herein.

TABLE 2

| Gene | Gleevec |
|---|---|
| ABL1 | 0.001 |
| JNK1A2/MAPK8 | 1.5 |
| JNK3A1/MAPK10 | 2 |
| LCK | 0.062 +/− 0.006 |
| PDGFRb | 0.02 |

48 kinase proteins were displayed on phage particle surfaces and assayed for their binding to BAY 43-9006. The binding constants (micromolar) are shown in Table 3, and the identifiers are as described above except VEGFR2 refers to the human form of vascular endothelial growth factor receptor-2 and is described herein.

TABLE 3

| Gene Symbol | BAY 43-9006 |
|---|---|
| ABL1 | 0.13 +/− 0.12 |
| ABL1(T334I) | 0.1 +/− 0.05 |
| p38/MAPK14 | 0.2 +/− 0.12 |
| PDGFRb | 0.041 +/− 0.009 |
| VEGFR2 | 0.07 +/− 0.06 |

Assay of Interactions Between Molecules and Target Proteins

The approach employs ATP-site dependent competition binding assays. The components are human kinases expressed as fusions to T7 bacteriophage particles and immobilized ligands that bind to the ATP site of one or more kinases, typically staurosporine. T7 phage replication leads to lysis of the bacterial host, and lysates containing tagged kinases are used in the assay. The immobilized small molecule ligands used to build the assays bind the kinases with high affinity (K_{d}<1 μM), and were amenable to attachment of biotin without disrupting binding. For the assay, tagged kinases and immobilized ATP site ligands are combined with the compound to be tested. If the test compound binds the kinase and directly or indirectly occludes the ATP site, it competes with the immobilized ligand and prevents binding to the solid support. If the compound does not bind the kinase, tagged proteins are free to bind to the solid support through the interaction between the kinase and the immobilized ligand. The results are read out by quantitating the amount of fusion protein bound to the solid support, which is accomplished with extraordinary sensitivity by either traditional phage plaque assays or by quantitative PCR (qPCR) using the phage genome as a template.

Kinases were cloned in a modified version of the commercially available T7 select 10-3 strain (Novagen). The head portion of each phage particle includes 415 copies of the major capsid protein, and in this system approximately one to ten of these are kinase fusion proteins. The N-terminus of the kinase is fused to the C-terminus of the capsid protein. The fusion proteins are randomly incorporated, and therefore distributed across the phage head surface.

To measure accurate K_{d}'s for test compounds the concentration of immobilized ligand was below the K_{d(probe)}, and the phage concentration was below the K_{d(probe)} and the K_{d(test)}. If both these concentrations were met the measured K_{d} for a test compound was independent of protein and immobilized ligand concentration. For each kinase assay we measured K_{d}'s for staurosporine or another appropriate inhibitor at two different phage concentrations and two different concentrations of immobilized ligand to confirm that these assumptions were met.

T7 phage grow to a titre of 10⁸ to 10¹⁰ pfu/mL. Each phage particle displays on average one to ten kinase molecules, and the concentration of phage-tagged kinase in the binding reaction is therefore in the low picomolar range. During the binding reaction the kinase can bind to either the test compound or the immobilized ligand. At low phage concentration the binding equilibrium equations yield the following expression for the binding constant of the interaction between the test compound and the kinase (K_{d(test)}): K_{d(test)}=(K_{d(probe)}/(K_{d(probe)}+[Probe]))×[test]_{1/2}. K_{d(probe)} is the binding constant for the interaction between the kinase and the immobilized ligand, [Probe] is the concentration of the immobilized ligand and [test]_{1/2} is the concentration of the test compound at the midpoint of the transition. The concentration of the immobilized ligand is kept in the low nanomolar range, below its binding constant for the kinase. Under these conditions the expression simplifies to K_{d(test)}=[test]_{1/2}, and K_{d(test)} is independent of the affinity of the immobilized ligand for the kinase (K_{d(probe)}).

Quantitative results of screening kinase inhibitors against protein kinases are shown in Tables 4, 5, and 6. Blank fields indicate combinations for which no evidence for binding was observed in a primary screen with a compound concentration of 10 μM. Numbers indicate binding constants (K_{d}'s) in μM. All binding constants are the average of at least two independent experiments.

TABLE 4

| | Gene Symbol | | |
|---|---|---|---|
| Accession # | (LocusLink) | Gene Symbol | BIRB-796 |
| XM_033355.1 | ABL1 | ABL1 | 1.5 |
| XM_033355 | ABL1 | ABL1(M370T) | 2.2 |
| XM_033355 | ABL1 | ABL1(Q271H) | 4.2 |
| XM_033355 | ABL1 | ABL1(T334I) | 0.041 |
| XM_033355 | ABL1 | ABL1(Y272F) | 2.3 |
| NM_004431.1 | EPHA2 | EPHA2 | 3.1 |
| NM_005233.2 | EPHA3 | EPHA3 | 0.58 |
| NM_004438.1 | EPHA4 | EPHA4 | 3.9 |
| NM_004439.3 | EPHA5 | EPHA5 | 1.3 |
| SK646 | EPHA6 | EPHA6 | 0.43 |
| NM_004440.1 | EPHA7 | EPHA7 | 0.22 |
| NM_020526.2 | EPHA8 | EPHA8 | 0.14 |
| NM_004441.2 | EPHB1 | EPHB1 | 4.2 |
| NM_002031.1 | FRK | FRK | 0.36 |
| NM_139068.1 | MAPK9 | JNK2 | 0.0056 |
| NM_002753.2 | MAPK10 | JNK3 | 0.062 |
| NM_005356.2 | LCK | LCK | 1.1 |
| NM_017572.1 | MKNK2 | MKNK2 | 1.1 |
| NM_002529.2 | NTRK1 | NTRK1 | 0.77 |
| NM_139012.1 | MAPK14 | p38-alpha | 0.00024 |
| NM_002751.4 | MAPK11 | p38-beta | 0.22 |
| NM_002969.2 | MAPK12 | p38-gamma | 0.014 |

TABLE 5

| | Gene Symbol | | |
|---|---|---|---|
| Accession # | (LocusLink) | Gene Symbol | Gleevec |
| XM_033355.1 | ABL1 | ABL1 | 0.0022 |
| XM_033355 | ABL1 | ABL1(E274K) | 0.11 |
| XM_033355 | ABL1 | ABL1(H415P) | 0.062 |
| XM_033355 | ABL1 | ABL1(M370T) | 0.014 |
| XM_033355 | ABL1 | ABL1(Q271H) | 0.024 |
| XM_033355 | ABL1 | ABL1(T334I) | 6.2 |
| XM_033355 | ABL1 | ABL1(Y272F) | 0.044 |
| NM_007314.1 | ABL2 | ABL2 | 0.013 |
| NM_002750.2 | MAPK8 | JNK1 | 3.2 |
| NM_139068.1 | MAPK9 | JNK2 | 5.2 |
| NM_002753.2 | MAPK10 | JNK3 | 3.3 |
| NM_005356.2 | LCK | LCK | 0.062 |
| NM_002609.2 | PDGFRB | PDGFRB | 0.028 |

TABLE 6

| | Gene Symbol | | |
|---|---|---|---|
| Accession # | (LocusLink) | Gene Symbol | BAY-43-9006 |
| XM_033355.1 | ABL1 | ABL1 | 0.13 |
| XM_033355 | ABL1 | ABL1(E274K) | 4.4 |
| XM_033355 | ABL1 | ABL1(H415P) | 1.2 |
| XM_033355 | ABL1 | ABL1(M370T) | 0.23 |
| XM_033355 | ABL1 | ABL1(Q271H) | 0.45 |
| XM_033355 | ABL1 | ABL1(T334I) | 0.17 |
| XM_033355 | ABL1 | ABL1(Y272F) | 0.58 |
| NM_007314.1 | ABL2 | ABL2 | 1.3 |
| NM_139012.1 | MAPK14 | p38-alpha | 0.26 |
| NM_002751.4 | MAPK11 | p38-beta | 0.2 |
| NM_002969.2 | MAPK12 | p38-gamma | 9.9 |
| NM_002609.2 | PDGFRB | PDGFRB | 0.041 |
| NM_002253.1 | KDR | VEGFR2 | 0.093 |

Assays for Gleevec-Resistant, Mutated Versions of the ABL Kinase

Resistance in most cases to Gleevec is due to either amplification of the BCR-ABL gene or to characteristic mutations in the ABL kinase that decrease sensitivity to Gleevec. To determine whether there are kinase inhibitors that are capable of inhibiting these therapeutically relevant mutated kinases we constructed tagged versions of six of the clinically observed mutant ABL kinases and screened kinase inhibitors for binding to these kinase variants.

The p38 inhibitor BIRB-796 binds ABL(T334I) with a ˜40 nM binding constant (see Table 5). This particular mutation is one of the most frequently observed in patients and the one most resistant to Gleevec. The results described here suggest the use of BIRB-796 as treatment for Gleevec-resistant CML.

Unless otherwise expressly stated, all terms of art, notations and other scientific terms or terminology used herein are intended to have the meanings commonly understood by those of skill in the art to which this invention pertains. The techniques and procedures described or referenced herein are commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of modulating an imatinib mesylate resistant tyrosine kinase activity comprising contacting a imatinib mesylate resistant tyrosine kinase polypeptide with an effective amount of BIRB-796.

2. The method of claim 1 wherein said imatinib mesylate resistant tyrosine kinase polypeptide is a Thr315Ile mutant of Abl tyrosine kinase.

3. The method of claim 1 wherein said contacting is performed in an animal subject and produces a beneficial effect on an imatinib mesylate resistant tyrosine kinase-mediated disease.

4. The method of claim 3 wherein said imatinib mesylate resistant tyrosine kinase-mediated disease is imatinib mesylate resistant chronic myelogenous leukemia.

5. A method of modulating a LCK kinase activity comprising contacting a LCK kinase polypeptide with an effective amount of imatinib mesylate.

6. The method of claim 5 wherein said contacting is performed in an animal subject and produces a beneficial effect on a LCK kinase-mediated disease.

7. The method of claim 6 wherein said LCK kinase-mediated disease is an inflammatory disorder and/or a disorder wherein an immunosuppression is desired.

8. A method of modulating a kinase activity comprising contacting a kinase polypeptide with an effective amount of BAY 43-9006 wherein said kinase polypeptide is at least one kinase selected from p38/MAPK14, imatinib mesylate resistant Abl kinase, imatinib mesylate sensitive Abl kinase, the platelet-derived growth factor receptor, and vascular endothelial growth factor receptor-2.

9. The method of claim 8 wherein said contacting is performed in an animal subject and produces a beneficial effect on a kinase-mediated disease, wherein said kinase-mediated disease is at least one disease selected from a p38/MAPK14-mediated disease, an imatinib mesylate resistant Abl kinase-mediated disease, an imatinib mesylate sensitive Abl kinase-mediated disease, a platelet-derived growth factor receptor-mediated disease, and a vascular endothelial growth factor receptor-2-mediated disease.

10. The method of claim 8 wherein said kinase-mediated disease is at least one disease selected from an inflammatory disorder, a chronic meylogenous leukemia, and a cancer.

11. The method of claim 1, 5, or 8 wherein said contacting is performed in vivo.

12. The method of claim 1, 5, or 8 wherein said contacting is performed in vitro.

13. A method of treating an imatinib mesylate resistant tyrosine kinase-mediated disease comprising administering to an animal subject in need thereof an effective amount of BIRB-796.

14. The method of claim 13 wherein said imatinib mesylate resistant tyrosine kinase-mediated disease is imatinib mesylate resistant chronic myelogenous leukemia.

15. A method of treating a LCK kinase-mediated disease comprising administering to an animal subject in need thereof an effective amount of imatinib mesylate.

16. The method of claim 15 wherein said LCK kinase-mediated disease is an inflammatory disorder and/or a disorder wherein an immunosuppression is desired.

17. A method of treating a kinase-mediated disease comprising administering to an animal subject in need thereof an effective amount of BAY 43-9006 wherein said kinase-mediated disease is at least one disease selected from a p38/MAPK14-mediated disease, an imatinib mesylate resistant Abl kinase-mediated disease, an imatinib mesylate sensitive Abl kinase-mediated disease, a platelet-derived growth factor receptor-mediated disease, and a vascular endothelial growth factor receptor-2-mediated disease.

18. The method of claim 17 wherein said kinase mediated disease is at least one disease selected from an inflammatory disorder, a cancer, and a disease wherein inhibition of smooth cell proliferation is desired.

19. The method of claim 17 wherein said kinase-mediated disease is a cancer and said cancer is treated by an inhibition of angiogenesis and/or prevention of growth of neovasculature.

20. The method of claim 17 wherein said kinase-mediated disease is at least one cancer selected from a solid tumor, a metasizeed tumor, an osteosarcoma, a small cell lung cancer, an angiomyolipoma, a neoplasm associated with tuberous sclerosis, and a myeloproliferative disease.

21. The method of claim 17 wherein said kinase-mediated disease is at least one disease selected from a diabetic retinopathy, a macular degeneration, and an ocular edema.

22. A method of treating hyperplasia and/or restenosis associated with vascular grafts comprising administering to an animal subject in need thereof an effective amount of BAY 43-9006.

23. A method of inhibiting angiogenesis and/or growth of neovasculature comprising administering to an animal subject in need thereof an effective amount of BAY 43-9006.

24. A method of treating inflammation and/or inducing immunosuppression comprising contacting a LCK kinase polypeptide with a compound that binds Bcr-Abl, c-kit, and PDGFR.

25. The method of claim 24 wherein said compound is imatinib mesylate.

26. A method of treating inflammation, psoriasis, and/or rheumatoid arthritis, said method comprising contacting a p38/MAPK14 polypeptide with a compound that binds Raf kinase, imatinib mesylate resistant or sensitive Abl kinase, PDGFR, and VEGFR2.

27. A method of treating angiogenesis comprising contacting a PDGFR and/or VEGFR2 polypeptide with a compound that binds Raf kinase, p38/MAPK14, and/or imatinib mesylate resistant or sensitive Abl kinase.

28. A method of treating smooth cell proliferation, intimal hyperplasia, and/or restenosis, said method comprising contacting a VEGFR2 polypeptide with a compound that binds Raf kinase, p38/MAPK14, imatinib mesylate resistant or sensitive Abl kinase, PDGFR, and VEGFR2.

29. The method of claim 25, 26, or 27 wherein said compound is BAY 43-9006.

30. A method of treating imatinib mesylate resitant chronic myelogenous leukemia comprising contacting an imatinib mesylate resistant Abl polypeptide with a compound that binds p38/MAPK14.

31. The method of claim 30 wherein said compound is BIRB-796.
